(19) United States
(12) Patent Application Publication
(10) Pub. No.: US 2026/0317793 A1
(43) Pub. Date: Oct. 15, 2026

(54) AN ENZYMATIC PROCESS FOR THE PRODUCTION OF VERBENONE AND IMPLEMENTATIONS THEREOF

(71) Applicants: ATGC BIOTECH PVT LTD, Hyderabad (IN); PHEROMONES BIOTECH LLP, Hyderabad (IN)

(72) Inventors: Srinivas Reddy Dumbala, Hyderabad (IN); Nitin Wasantrao Fadnavis, Hyderabad (IN); Narasimha Rao Nizampatnam, Hyderabad (IN); Ravindra Babu Peram, Hyderabad (IN); Vijay Bhasker Reddy Lachagari, Hyderabad (IN); Sivaram Pasad Lekkala, Hyderabad (IN); Akshitha Reddy Kaukuttla, Hyderabad (IN); Markandeya Gorantla, Hyderabad (IN)

(21) Appl. No.: 19/485,250

(22) PCT Filed: May 9, 2024
(86) PCT No.: PCT/IN2024/050506

§ 371 Date: Nov. 17, 2025

(30) Foreign Application Priority Data
May 18, 2023 (IN) 202341034948

(51) Int. Cl. C12P 7/26; C12N 9/02; C12N 9/04; C12N 9/08

(52) U.S. Cl. CPC C12P 7/26; C12N 9/0006; C12N 9/0061; C12N 9/0065; C12Y 101/03004; C12Y 110/03002; C12Y 111/01006

(57) ABSTRACT

The present disclosure provides a process for the conversion of α-pinene to verbenone comprising: (a) mixing α-pinene and an aqueous solution comprising laccase, at a temperature in the range of 20° C. and 45° C. to obtain a reaction mixture; and (b) adding a mediator to the reaction mixture in the presence of an oxygen source, to obtain verbenone.

### FIELD OF INVENTION

The present disclosure relates to the field of pheromone production. The present disclosure in particular relates to the process of enzymatic conversion of α-pinene to verbenone.

### BACKGROUND OF THE INVENTION

Allylic oxidations of olefins, e.g., cyclohexene, to enones, e.g., cyclohex-2-en-1-one, are valuable organic transformations. Allylic oxidation allows the conversion of alkenes (α-pinene, 1) into allyl alcohols (3 and 4) or enones (verbenone, 2) and thus, permit the synthesis of value-added products, such as pheromones from simple precursor (scheme 1).

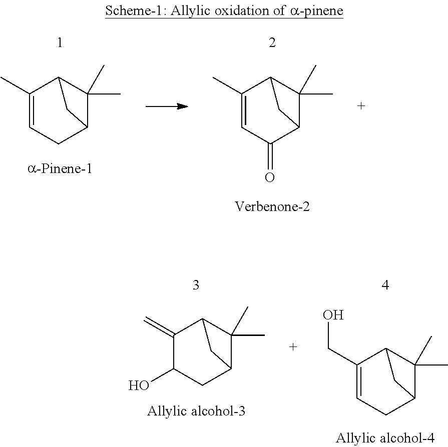

The required olefinic starting materials are readily available as cheap bulk chemicals and many interesting derivatives, such as terpenes are available from renewable sources. Therefore, allylic oxidations are particularly attractive in natural products chemistry and the conversion of terpenoids into allyl α-pinenes and enones is of high economic and scientific interest. The resulting oxidation products are attractive synthetic intermediates, or they may be used directly, for example, as flavour compounds, pheromones, or pharmaceuticals.

A variety of stoichiometric and catalytic metal-based methods are available for these conversions. For example, allylic oxidations can be carried out with reagents based on several metals such as chromium, copper, rhodium, selenium, cobalt, ruthenium, palladium, iron etc. Other metal reagents such as potassium permanganate, manganese acetate are also used in the conversion process. Owing to the toxicity of many transition metals, such as chromium, the high cost of metals such as ruthenium or palladium, and problematic separation of the product from trace metallic impurities, metal-catalyzed allylic oxidations are often unattractive from the industrial and environmental points of view. This is particularly true for the food and pharmaceutical industries. In contrast, biocatalytic approaches have achieved a growing interest in recent years due to better selectivity and environmental friendliness.

α- and β-pinene represent 75 to 90% of essential oils from conifers and can be found in concentrations in the range from 50 to 70% and from 15 to 30%, respectively, in turpentine, a by-product of the paper and cellulose industry. These compounds are the most abundant bicyclic monoterpenes and can be precursors of aroma compounds of impact that are widely used in cosmetic and food industries, such as α-terpineol, verbenol, and verbenone. Although, α- and β-pinene are promising substrates, the physicochemical characteristics of these compounds represent challenges that require efforts for their effective application for biotransformation, such as their chemical instability, volatility, and toxicity to microbial cells. In addition, the low solubility of α- and β-pinene (0.026 and 0.049 mmol/L, respectively) results in a low availability of the substrate to the biocatalyst.

Over the past few decades, many biotransformation processes of α-pinene to verbenone have been reported using fungi and plant species. Although, academically interesting, these routes are not industrially viable since the substrate concentrations are very dilute (<0.1%). Product extraction and purification in such cases is highly uneconomical.

Therefore, there is a need for developing an effective process for the bioconversion of α-pinene, particularly for production of industrially important chemicals, such as verbenone, a pheromone. Further, it is desirable that the process is simple, industrially scalable, and economical.

### SUMMARY

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

In an aspect of the present disclosure, there is provided a process for the conversion of α-pinene to verbenone comprising: (a) mixing α-pinene and an aqueous solution comprising laccase, at a temperature in a range of 20° C. to 45° C., to obtain a reaction mixture; and (b) adding a mediator to the reaction mixture in the presence of an oxygen source, to obtain verbenone.

In an aspect of the present disclosure, there is provided a process for the conversion of α-pinene to verbenone comprising: (a) mixing the α-pinene and an aqueous solution comprising laccase and catalase, at a temperature in the range of 20° C. and 45° C., to obtain a reaction mixture, wherein the weight ratio of laccase and α-pinene is in the range of 1.0:0.5 to 1.0:5.0; (b) adding a mediator, and an emulsifying agent to the reaction mixture in the presence of hydrogen peroxide under stirring for a period in the range of 10 to 72 hours to obtain verbenone, wherein the activity ratio of laccase and catalase is in the range of 1.0:1.0 to 10.0:1.0.

In an aspect of the present disclosure, there is provided a process for the conversion of α-pinene to verbenone comprising: (a) mixing the α-pinene and an aqueous solution comprising laccase, catalase, and glucose oxidase at a temperature in the range of 20° C. and 45° C., to obtain a reaction mixture, wherein the weight ratio of laccase and α-pinene is in the range of 1.0:0.5 to 1.0:5.0; (b) adding a mediator and an emulsifying agent in the presence of glucose to the reaction mixture under stirring for a period of 10 to 72 h to obtain verbenone, wherein the activity ratio of laccase, catalase and glucose oxidase is in the range of 1.0:1.0:0.5 to 1.0:1.0:0.1.

### BRIEF DESCRIPTION OF DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the Gas Chromatogram (GC) for the conversion product of verbenone from α-pinene using laccase, in accordance with an embodiment of the present disclosure.

FIG. 2 depicts the GC for the conversion product of verbenone from α-pinene using laccase and catalase, in accordance with an embodiment of the present disclosure.

FIG. 3 depicts the GC for the conversion product of verbenone from α-pinene using laccase, catalase, and glucose oxidase, in accordance with an embodiment of the present disclosure.

FIG. 4 depicts the GC for the conversion product of verbenone from α-pinene using PEG coated laccase, in accordance with an embodiment of the present disclosure.

FIG. 5 depicts the GC of 99% pure verbenone obtained by the bioconversion of α-pinene using laccase, in accordance with an embodiment of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Terms

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those skilled in the art, however, for convenience and completeness, particular terms and their meanings are set forth below.

The articles “a”, “an” and “the” are used to refer to one or to more than one (i.e., to at least one) of the grammatical object of the article.

Throughout this specification, unless the context requires otherwise the word “comprise”, and variations such as “comprises” and “comprising” are used in the inclusive, open sense, and will be understood to imply the inclusion of a stated element or step or group of element or steps but not the exclusion of any other element or step or group of element or steps. It is not intended to be construed as “consists of only”.

The term “including” is used herein to mean “including but not limited to”. “Including” and “including but not limited to” are used interchangeably.

The term “biocatalysts”, as used herein, refers to biological systems or substances capable of catalyzing reactions. Biological systems may be whole bacterial cell or yeast cell or their components, substances include enzymes. In an example of the present disclosure, biocatalysts are enzymes. Examples of enzymes include, but are not limited to laccase, catalase, glucose oxidase, or combinations thereof.

The term “vvm”, as used herein refers to the volume of air bubbled per unit volume of a medium per minute, which is calculated by dividing measured airflow rate (L/m) with the volume (L) of medium.

The term “activity ratio of enzymes” refers to the ratio between the activity of each enzyme used in a process, which is measured in units that indicate the rate of reaction catalyzed by that enzyme expressed as micromoles of substrate transformed or product formed.

The term “yield”, as used herein refers to the conversion efficiency of a substrate to a product in a process.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described. All publications mentioned herein are incorporated herein by reference.

The present disclosure is not to be limited in scope by the specific embodiments described herein, which are intended for the purposes of exemplification only. Functionally equivalent products, compositions, and methods are clearly within the scope of the disclosure, as described herein.

The present disclosure provides a process for the production of pheromones, such as verbenone from α-pinene. The process includes the use of a combination of enzymes to achieve the selective and effective conversion of α-pinene to verbenone. The process, according to embodiments herein, achieves the production of verbenone at a purity of about 99%. Further, according to the present disclosure, the process is highly selective, owing to the use of enzymes, and results in about 89% conversion of α-pinene to verbenone. The disclosed process, therefore, involves simple downstream purification processes, scalable to industrial standards, and is economical.

### Process for the Enzymatic Conversion of α-Pinene to Verbenone

In an embodiment of the present disclosure, there is provided a process for the enzymatic conversion of α-pinene to verbenone.

In an embodiment, there is provided a process for the conversion of α-pinene to verbenone comprising: (a) mixing α-pinene and an aqueous solution comprising laccase, at a temperature in a range of 20° C. to 45° C., to obtain a reaction mixture; and (b) adding a mediator to the reaction mixture in the presence of an oxygen source, to obtain verbenone.

In an embodiment, there is provided a process for the conversion of α-pinene to verbenone comprising: (a) mixing α-pinene and an aqueous solution comprising laccase, at a temperature in a range of 20° C. to 45° C., to obtain a reaction mixture; and (b) adding a mediator to the reaction mixture in the presence of an oxygen source, to obtain verbenone; wherein the weight ratio of laccase and α-pinene is in a range of 1.0:0.5 to 1.0:5.0.

The α-pinene, according to embodiments herein, is procured commercially. In an embodiment, the α-pinene is mixed with an aqueous solution of enzyme capable of catalyzing the oxidation of α-pinene in the presence of an oxygen source and a mediator.

In an embodiment, the α-pinene is in a weight range of 0.5 to 50%. In another embodiment, the α-pinene is in a weight range of 1 to 30%. In yet another embodiment, the α-pinene is in a weight range of 2 to 20%.

The enzyme, according to embodiments herein, is a biocatalyst capable of catalyzing the oxidation of α-pinene in the presence of an oxygen source and a mediator.

In an embodiment, the enzyme is selected from laccase, catalase, glucose oxidase, or combinations thereof.

In one embodiment, the enzyme is laccase, which is a multi-copper oxidase that catalyzes monoelectronic oxidation of a wide range of organic compounds and substrates.

In some embodiments, the enzyme is a combination of laccase and catalase. In an embodiment, the activity ratio of laccase and catalase is in the range of 1:1 to 10:1.

In some embodiments, the enzyme is a combination of laccase, catalase, and glucose oxidase. In an embodiment, the activity ratio of laccase, catalase, and glucose oxidase is in the range of 1.0:1.0:0.5 to 1.0:1.0:0.1.

According to the embodiments herein, the laccase is obtained from commercial sources. Laccase enzymes are commonly produced by fungi, and also by some bacteria and plants. Examples of fungi include, but are not limited to, Trametes versicolor, Trametes pubescens, Pyricularia oryzae, or Agaricus bisporus, Mycelopthora termophila. Various methods are known in the art to produce laccase from fungal species. Embodiments herein may include laccase produced from any such fungal species. Alternatively, laccases can be obtained by the heterogenous expression of laccases in other microbes.

Catalases are enzymes that are commonly found in all aerobic organisms. Catalases catalyze the decomposition of hydrogen peroxide into water and oxygen. Therefore, hydrogen peroxide serves as one of the oxygen sources in the process disclosed herein. Various methods are known in art to produce catalases from various organisms. Embodiments herein may include catalases produced from any such organisms. In a preferable embodiment, the catalase is obtained from commercial sources.

Glucose oxidase is an oxidoreductase enzyme produced by certain species of fungi and insects. Glucose oxidase catalyzes the oxidation of β-D-glucose to D-glucono-δ-lactone and hydrogen peroxide. The hydrogen peroxide is further decomposed by the catalase enzyme into water and oxygen as disclosed herein. Therefore, glucose serves as one of the oxygen sources in the process disclosed herein. Various methods are known in art to produce glucose oxidase from various organisms. Embodiments herein may include glucose oxidase produced from any such organisms. In a preferable embodiment, the glucose oxidase is obtained from commercial sources.

In some embodiments, the laccase is subjected to further modification in order to improve its stability and activity of laccase. Various methods are known in the art for the modification of laccases. According to embodiments herein, the laccase is modified by extracting into polyethylene glycol (PEG), to obtain PEG coated laccase. In an embodiment, the PEG is selected from PEG-200, PEG-400, or combinations thereof.

In some embodiments, the laccase or the PEG coated laccase is dissolved in an aqueous solution.

In some embodiments, the enzymes, laccase, and catalase are dissolved in an aqueous solution.

In some embodiments, the enzymes, laccase, catalase, and glucose oxidase are dissolved in an aqueous solution.

In an embodiment, the laccase is in a weight range of 0.05 to 15.0%. In another embodiment, the laccase is in a weight range of 0.1 to 10.0%. In yet another embodiment, the laccase is in a weight range of 0.5 to 5.0%.

In an embodiment, the catalase is in a weight range of 0.01 to 10%. In another embodiment, the catalase is in a weight range of 0.01 to 5%. In yet another embodiment, the catalase is in a weight range of 0.1 to 2.0%.

In an embodiment, the glucose oxidase is in a weight range of 0.005 to 5%. In another embodiment, the glucose oxidase is in a weight range of 0.01 to 1.0%. In yet another embodiment, the glucose oxidase is in a weight range of 0.05 to 0.50%.

In some embodiments of the present disclosure, the aqueous solution comprises a buffer selected from tris-HCl buffer, sodium citrate buffer, sodium phosphate buffer, potassium phosphate buffer, sodium or potassium acetate buffers, or combination thereof; and the aqueous solution is maintained at a pH in the range of 2.5 to 6.5.

In some embodiments, the aqueous solution is maintained at a suitable pH, to maintain the stability and activity of the enzymes. In an embodiment, the aqueous solution is maintained at a pH in the range of 2.0 to 8.5. In another embodiment, the aqueous solution is maintained at a pH in the range of 2.5 to 7.5. In yet another embodiment, the aqueous solution is maintained at a pH in the range of 2.5 to 6.5.

In some embodiments, the buffer used herein is of a suitable molarity to maintain the pH of the aqueous solution at a desired range as described herein. In an embodiment, the buffer molarity is in the range of 0.01 to 2.0 M. In another embodiment, the buffer molarity is in the range of 0.02 to 1.5 M. In yet another embodiment, the buffer molarity is in the range of 0.1 to 1.0 M.

In an embodiment, the step (a) of the disclosed process comprises mixing the α-pinene and an aqueous solution comprising laccase, at a temperature in the range of 10° C. and 65° C. In another embodiment, the temperature is in the range of 15 to 50° C. In yet another embodiment, the temperature is in the range of 20 to 45° C.

In an embodiment, the step (a) of the disclosed process comprises mixing the α-pinene and an aqueous solution comprising laccase, at a temperature in the range of 20° C. and 45° C., wherein the weight ratio of laccase and α-pinene is in the range of 1.0:0.5 to 1.0:5.0 to obtain a reaction mixture.

In an embodiment of the present disclosure, an oxygen source is provided to the reaction mixture obtained from step (a) of the process disclosed herein.

The term “oxygen source” used herein refers to a chemical reagent that provides an oxo group for the allylic oxidation of α-pinene. According to embodiments herein, the oxygen source is selected from air, hydrogen peroxide, glucose, or combinations thereof.

In one embodiment, the oxygen source provided to the reaction mixture is air, which is bubbled through the reaction mixture at a rate of 0.001 vvm to 0.5 vvm. In another embodiment, air is bubbled through the reaction mixture at a rate of 0.001 vvm to 0.1 vvm. In yet another embodiment, air is bubbled through the reaction mixture at a rate of 0.001 vvm to 0.01 vvm.

In some embodiments, the oxygen source provided to the reaction mixture is hydrogen peroxide. In an embodiment, hydrogen peroxide is in a weight range of 0.001 to 20%. In another embodiment, hydrogen peroxide is in a weight range of 0.001 to 10%. In yet another embodiment, hydrogen peroxide is in a weight range of 0.001 to 1%.

In some embodiments, the oxygen source provided to the reaction mixture is glucose. In an embodiment, glucose is in a weight range of 0.001 to 5%. In another embodiment, glucose is in a weight range of 0.001 to 2%. In yet another embodiment, glucose is in a weight range of 0.001 to 0.5%.

In an embodiment of the present disclosure, in step (b) of the disclosed process, a mediator is added to the reaction mixture in the presence of an oxygen source.

The term “mediator”, as used herein, refers to the electron transfer reagents, which acts as a sort of ‘electron shuttle’, to initiate the allylic oxidation of α-pinene. For the reactions where the substrate to be oxidized has a redox potential higher than laccase, or the substrate is too large to penetrate the enzyme active site, the presence of a low-molecular weight chemical mediator is required to facilitate oxidative reactions. A mediator acts as a sort of ‘electron shuttle’; once it is oxidized by laccase, it diffuses away from the enzymatic pocket and in turn oxidizes any substrate that, due to its size, could not directly enter the enzymatic pocket. By using these so called ‘chemical mediators’, the redox potential of laccase can be extended which allows the oxidation of the substrate (Scheme 2).

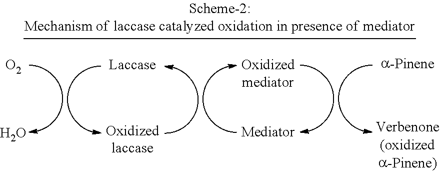

Various mediators are known in the art and can be used in the process described herein. In an embodiment, the mediator is selected from (2,2,6,6-tetramethylpiperidin-1-yl)oxyl (TEMPO), hydroxybenzotriazole (HOBT), dimethylglyoxime, N-hydroxyphthalimide (NHPI), 4-acetamido-TEMPO, TEMPO-4-amino-4-carboxylic acid, 4-hydroxy-TEMPO, 4-hydroxy-TEMPO-benzoate, 4-oxo-TEMPO, 4-methoxy-TEMPO, 4-amino-2,2,6,6,-tetramethylpiperidine, 4-amino-TEMPO or combinations thereof.

In an embodiment, the mediator is in a weight range of 0.001 to 5.5%. In another embodiment, the mediator is in a weight range of 0.01 to 3%. In yet another embodiment, the mediator is in a weight range of 0.2 to 2.0%.

In an embodiment, the mole ratio of mediator and α-pinene is in a range of 1.0:5.0 to 1.0:15.0.

Embodiments herein include the addition of an emulsifying agent to the reaction mixture in step (b) of the disclosed process.

The term “emulsifying agent”, as used herein, refers to a chemical compound that maintains the stability of emulsions and prevents the separation of water and oil phases. Emulsifying agent used herein ensures the solubility of the enzymes and the substrate in the reaction medium. Various emulsifying agents are known in the art and can be used in various embodiments herein. In an embodiment, the emulsifying agent is a non-ionic surfactant selected from polyethylene glycol (PEG)-200, PEG-400, 2-[4-(2,4,4-trimethylpentan-2-yl) phenoxy]ethanol (triton-X 100), polyoxyethylene (80) sorbitan monooleate (tween 80), or combinations thereof. In another embodiment, the emulsifying agent is polyethylene glycol (PEG) having molecular weight in a range of 200 to 2000, preferably in a range of 200 to 600.

In an embodiment, the emulsifying agent used herein is in a weight range of 0.01 to 50.0%. In another embodiment, the emulsifying agent used herein is in a weight range of 0.1 to 10%. In yet another embodiment, the emulsifying agent used herein is in a weight range of 0.1 to 5.0%.

In an embodiment of the present disclosure, the step (b) of the disclosed process is carried out under stirring for a period in the range of 10 to 72 h to obtain verbenone. In another embodiment of the present disclosure, the step (b) of the disclosed process is carried out under stirring for a period in the range of 10 to 50 h to obtain verbenone. In yet another embodiment of the present disclosure, the step (b) of the disclosed process is carried out under stirring for a period in the range of 20 to 30 h to obtain verbenone.

In some embodiments, the disclosed process is carried out in a stirred tank reactor or an autoclave under oxygen pressure.

In an embodiment, the disclosed process is carried out in an autoclave under oxygen or air pressure ranging from 0.1 kgf/cm² to 10 kgf/cm². In another embodiment, the disclosed process is carried out in an autoclave under oxygen or air pressure ranging from 0.1 kgf/cm² to 5.0 kgf/cm². In yet another embodiment, the disclosed process is carried out in an autoclave under oxygen or air pressure ranging from 0.1 kgf/cm² to 2.0 kgf/cm².

In an embodiment, there is provided a process for the conversion of α-pinene to verbenone comprising: (a) mixing the α-pinene and an aqueous solution comprising laccase, at a temperature in the range of 20° C. and 45° C., to obtain a reaction mixture, wherein the weight ratio of laccase and α-pinene is in the range of 1.0:0.5 to 1.0:5.0; (b) adding a mediator and an emulsifying agent to the reaction mixture in the presence of an oxygen source, under stirring for a period in the range of 10 to 72 hours to obtain verbenone.

In another embodiment, there is provided a process for the conversion of α-pinene to verbenone comprising: (a) mixing the α-pinene and an aqueous solution comprising laccase and catalase, at a temperature in the range of 20° C. and 45° C., to obtain a reaction mixture, wherein the weight ratio of laccase and α-pinene is in the range of 1.0:0.5 to 1.0:5.0; (b) adding a mediator, and an emulsifying agent, to the reaction mixture in the presence of hydrogen peroxide under stirring for a period in the range of 10 to 72 h to obtain verbenone, wherein the activity ratio of laccase and catalase is in the range of 1.0:1.0 to 10.0:1.0.

In yet another embodiment there is provided a process for the conversion of α-pinene to verbenone comprising: (a) mixing the α-pinene and an aqueous solution comprising laccase, catalase, and glucose oxidase at a temperature in the range of 20° C. and 45° C., to obtain a reaction mixture, wherein the weight ratio of laccase and α-pinene is in the range of 1.0:0.5 to 1.0:5.0; (b) adding a mediator and an emulsifying agent in the presence of glucose to the reaction mixture under stirring for a period of 10 to 72 h to obtain verbenone, wherein the activity ratio of laccase, catalase and glucose oxidase is in the range of 1.0:1.0:0.5 to 1.0:1.0:0.1.

### Extraction of Verbenone

Embodiments herein include the extraction of the reaction mixture obtained from step (b) of the process disclosed herein to obtain verbenone.

In some embodiments, the verbenone is obtained by extracting the reaction mixture using a solvent. In an embodiment, the solvent is selected from hexane, heptane, ethyl acetate, methyl tertiary butyl ether, methyl isobutyl ketone, or combinations thereof.

In an embodiment, the amount of solvent used for extraction is in the range of 0.1 to 2.0 volumes of reaction mixture.

In an embodiment, the yield of the verbenone obtained from the process disclosed herein is in a range of 50% to 80%.

### Purification of Verbenone

Embodiments herein include the further purification of verbenone obtained from step (b) of the process disclosed herein. Various methods for purification of verbenone are known in the art and can be used in the process described herein. In an embodiment, the verbenone is purified using sodium bisulphite.

In an embodiment, the purity of the verbenone obtained from the process disclosed herein is in a range of 95% to 99%. In another embodiment, the purity of the verbenone obtained from the process disclosed herein is in a range of 96% to 99%. In another embodiment, the purity of the verbenone obtained from the process disclosed herein is in a range of 97% to 99%.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar to or equivalent to those described herein can be used in the practice of the disclosed methods and compositions, the exemplary methods, devices, and materials are described herein. It is to be understood that this disclosure is not limited to methods, and experimental conditions described, as such methods and conditions may apply.

### Materials

The chemicals such as TEMPO, PEG 400, PEG 200 were procured from Sisco Research Laboratories Pvt. Ltd (SRL) India.

The laccase enzyme was procured from M/s Aumgene Biosciences, or Americos Chemicals Pvt Ltd, India.

The enzymes, such as catalase and glucose oxidase was procured from M/s Biolaxi Corporation.

### Example 1

Preparation of Verbenone with Laccase

The commercially (Aumgene Biosciences) available laccase (1.0 g, uncoated laccase) was dissolved in distilled water or citrate buffer (0.1 M, 100 mL), and 1.0% emulsifying agent was added. α-pinene (2.20 g) was added to the enzyme solution (aqueous solution) and stirred at 35° C. while passing air (oxygen source) through the reaction mixture. The solution of TEMPO (mediator, 0.25 g in 10 mL distilled water), in water was added and the reaction mixture was stirred for 24 h with bubbling of air (0.001 vvm). The product was extracted twice with 20 mL of hexane (solvent) and recovered after evaporation of hexane.

Gas Chromatography (GC) was used to determine the conversion percentage of verbenone from α-pinene. One microliter of reaction mixture was analyzed on an Agilent 7890B series gas chromatograph coupled to a mass-selective detector Agilent 5977B (Agilent Technologies, California, United States). The GC was equipped with a HP-INNOWax capillary column (30 m×250 μm×0.25 μm film thickness). Helium was the carrier gas (velocity 20 mL/min) and the injector was configured in split mode and maintained at 250° C. injector temperature, 1 mL/min column flow. In order to obtain good separation, the oven temperature was held at 50° C. for 5 min and rose at a rate of 10° C./min up to 160° C. and was held for 5 min, at a rate of 5° C./min up to 220° C. and held for 5 min, at a rate of 10° C./min up to 260° C. and with a final hold for 10 min.

### Result

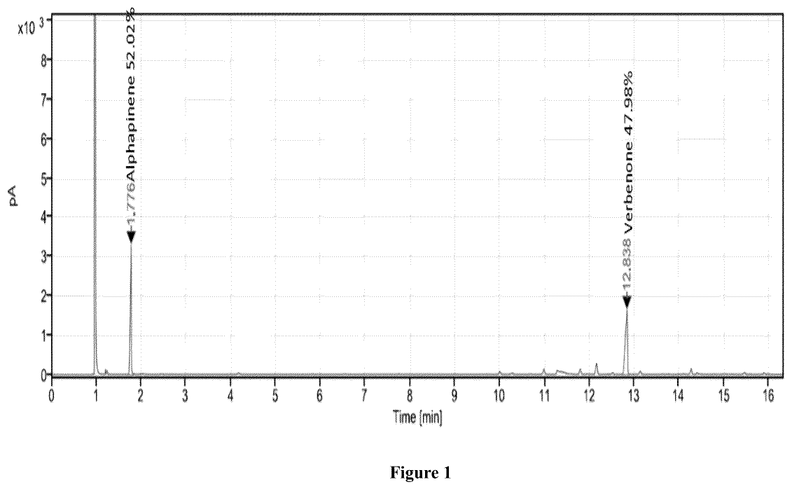

The GC analysis of the reaction mixture showed 47.98% conversion of α-pinene to verbenone (refer table 1 of FIG. 1).

TABLE 1

| Signal: | FID1A | | | | | |
|---|---|---|---|---|---|---|
| RT | Compound | | Width | | | Area |
| [min] | Name | Type | [min] | Area | Height | % |
| 1.776 | Alpha-pinene | MM m | 0.13 | 5838.08 | 3302.32 | 52.02 |
| 12.838 | Verbenone | MM m | 0.18 | 5383.88 | 1642.64 | 47.98 |
| | | | Sum | 11221.96 | | |

The table 1 represents the data of GC analysis of the reaction mixture of FIG. 1.

### Example 2

Preparation of Verbenone with Laccase Coupled with Catalase and Hydrogen Peroxide

The commercially available laccase (1.0 g), and catalase (Biolaxi Enzymes) (1.0 g), in the activity ratio of 1.0:1.0 were dissolved in distilled water or in citrate buffer (0.1M, 100 mL). α-pinene (2.20 g) was added to the enzyme solution (aqueous solution) and emulsifying agent (1.0%) was added, stirred at 35° C. The solution of TEMPO (0.25 g in 10 mL distilled water, mediator), was added. 1M solution of hydrogen peroxide (oxygen source) was added with a dosing pump at a rate of 0.1 mL/min and the reaction mixture was stirred for 24 h. The product was extracted twice with 20 mL of hexane (solvent) and recovered after evaporation of hexane. The GC analysis of the reaction mixture was performed as described earlier in Example 1.

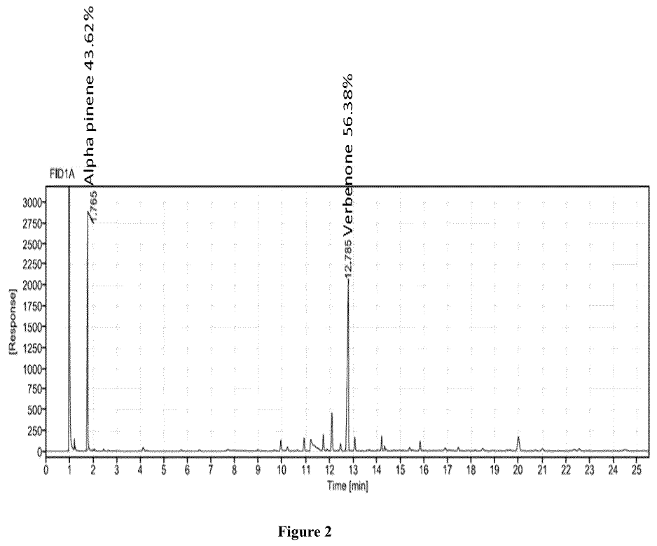

The GC analysis of the reaction mixture showed 56.38% conversion of α-pinene to verbenone (refer table 2 of FIG. 2).

TABLE 2

| Signal: | FID1A | | | | | |
|---|---|---|---|---|---|---|
| RT | | Width | | | Area | |
| [min] | Type | [min] | Area | Height | % | Name |
| 1.765 | MM m | 0.12 | 5732.72 | 2853.89 | 43.62 | |
| 12.785 | MM m | 0.19 | 7408.94 | 2005.69 | 56.38 | |

The table 2 represents the data of GC analysis of the reaction mixture of FIG. 2.

### Example 3

Preparation of Verbenone with Laccase Coupled with Catalase and Glucose Oxidase

The commercially available enzymes laccase (1.0 g), catalase (1.0 g) and glucose oxidase (Biolaxi Enzymes) (0.5 g) in the activity ratio of 1.0:1.0:0.5 were dissolved in citrate buffer (0.1 M) (100 mL), and 1.0% emulsifying agent was added. α-pinene (2.20 g) was added to the enzyme solution (aqueous solution) and solution of TEMPO (0.25 g in 10 mL distilled water, mediator) in water was added, stirred at 35° C. A 10% solution of glucose (oxygen source) in water was added to the reaction with a dosing pump at a rate of 0.1 mL/min and the reaction mixture was stirred for 24 h. The product was extracted twice with 20 mL of hexane (solvent) and recovered after evaporation of hexane. The GC analysis of the reaction mixture was performed as described earlier in Example 1.

### Results

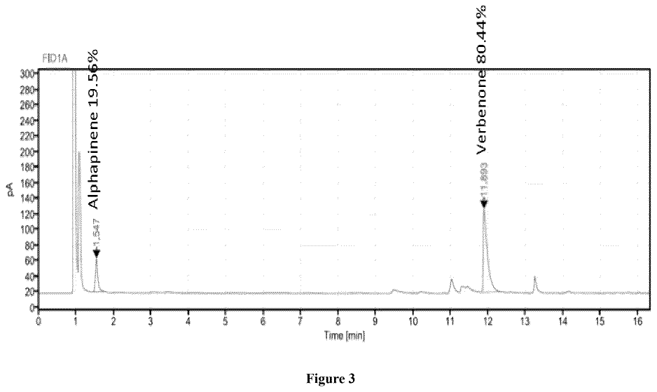

The GC analysis of the reaction mixture showed 80.44% conversion of α-pinene to verbenone (refer table 3 of FIG. 3).

TABLE 3

| Signal: | FID1A | | | | | |
|---|---|---|---|---|---|---|
| RT | Compound | | Width | | | Area |
| [min] | Name | Type | [min] | Area | Height | % |
| 1.547 | Alpha-pinene | MM m | 0.37 | 202.15 | 42.62 | 19.56 |
| 11.893 | Verbenone | MM m | 0.51 | 831.46 | 106.54 | 80.44 |
| | | | Sum | 1033.62 | | |

The table 3 represents the data of GC analysis of the reaction mixture of FIG. 3.

### Example 4

Preparation of Verbenone with Laccase in PEG-400

Laccase was extracted with PEG (emulsifying agent) to improve its stability.

### Extraction of Laccase in PEG-400

10 g laccase was dissolved in 100 mL water, 12 g K₂HPO₄ and 4 g KH₂PO₄ was added to the laccase solution. 100 mL of PEG-400 was further added, and the mixture was stirred for 4 h at RT. After centrifugation, the PEG coated laccase was separated (recovered ~160 mL) and stored in refrigerator.

The above PEG coated laccase (20 mL, based on recovery and activity is equivalent to 1 g of laccase) was mixed with a citrate buffer (0.1 M, 80 mL) and the pH was adjusted to 3.6. α-pinene (2.20 g) and TEMPO (0.25 g, mediator) were added, and the reaction mixture was stirred at 35° C. for 24 h with bubbling of air (0.001 vvm). The product was extracted twice with 20 mL of hexane (solvent) and recovered after evaporation of hexane. The GC analysis of the reaction mixture was performed as described earlier in Example 1.

### Results

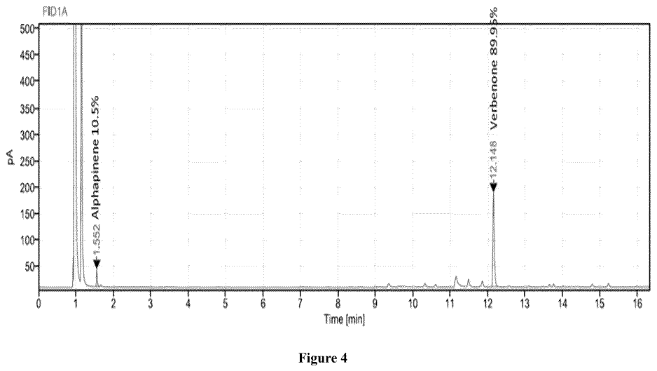

The conversion of α-pinene to verbenone after 24 hours was 89.95% using PEG coated laccase (refer table 4 of FIG. 4).

TABLE 4

| Signal: | FID1A | | | | | |
|---|---|---|---|---|---|---|
| RT | Compound | | Width | | | Area |
| [min] | Name | Type | [min] | Area | Height | % |
| 1.552 | Alpha-pinene | MM m | 0.11 | 54.07 | 32.00 | 10.05 |
| 12.148 | Verbenone | MM m | 0.32 | 483.83 | 177.80 | 89.95 |
| | | | Sum | 537.90 | | |

The table 4 represents the data of GC analysis of the reaction mixture of FIG. 4.

### Example 5

### Purification of Verbenone

The reaction mixture obtained in examples 1~4 after removal of the solvent (10 g) was suspended in water (100 mL) and added dropwise to a solution of sodium bisulfite (16 g) in water (30 mL) at room temperature. The resulting mixture was stirred for 6 h, wherein verbenone formed a bisulfite adduct and was dissolved in water. The reaction mixture was then extracted with methyl tertiary butyl ether (MTBE). The washings were pooled, and the organic solvent was removed on rotavapor to collect unreacted α-pinene.

The washed bisulfite adduct was cooled in an ice bath. 6N HCl was added dropwise to give a clear aqueous layer with an organic layer of verbenone. The verbenone was extracted with hexane, washed with water, and dried over anhydrous magnesium sulfate.

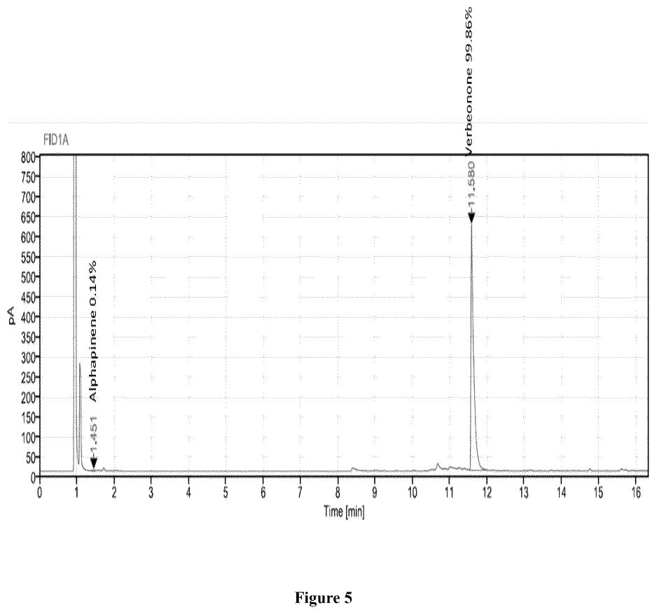

Removal of the solvent on rotavapor resulted in verbenone with 99% purity and yield obtained was 70% (refer table 5 of FIG. 5).

TABLE 5

| Signal: | FID1A | | | | | |
|---|---|---|---|---|---|---|
| RT | Compound | | Width | | | Area |
| [min] | Name | Type | [min] | Area | Height | % |
| 1.451 | Alpha-pinene | MM m | 0.13 | 4.32 | 1.63 | 0.14 |
| 11.580 | Verbenone | MM m | 0.57 | 3160.14 | 616.26 | 99.86 |

The table 5 represents the data of GC analysis of the reaction mixture of FIG. 5.

### Advantages Of The Present Disclosure

The present disclosure provides a process to produce pheromones, such as verbenone from α-pinene. Conversion of α-pinene to verbenone is carried out using enzymes, such as laccase, catalase, glucose oxidase, or combinations thereof. The disclosed process exhibits the following advantages.

Verbenone obtained is of high (99%) purity.

The process is cost-effective and environmentally friendly, owing to the use of biocatalysts.

The process is carried out at optimal temperature and does not involve the use of harsh chemicals.

The process resulted in higher conversion (i.e., 89%) of α-pinene when compared to conventional chemical processes.

The process is highly selective and does not result in any byproducts when compared to the conventional chemical processes.

What is claimed is:

1. A process for the conversion of α-pinene to verbenone comprising:
(a) mixing α-pinene and an aqueous solution comprising laccase, at a temperature in a range of 20° C. to 45° C., to obtain a reaction mixture; and
(b) adding a mediator to the reaction mixture in the presence of an oxygen source, to obtain verbenone.

2. The process as claimed in claim 1, wherein the weight ratio of laccase and α-pinene is in a range of 1.0:0.5 to 1.0:5.0.

3. The process as claimed in claim 1, wherein the step (b) comprises adding an emulsifying agent to the reaction mixture.

4. The process as claimed in claim 1, wherein the step (b) is carried out under stirring for a period in a range of 10 to 72 hours.

5. The process as claimed in claim 1, wherein the aqueous solution comprises a buffer selected from tris-HCl buffer, sodium citrate buffer, sodium phosphate buffer, potassium phosphate buffer, sodium acetate buffer, potassium acetate buffer, or combinations thereof; and the aqueous solution is maintained at a pH in the range of 2.5 to 6.5.

6. The process as claimed in claim 1, wherein the process comprises extracting and purifying said verbenone by treating with sodium bisulphite.

7. The process as claimed in claim 1, wherein the laccase is uncoated laccase, or a polyethylene glycol (PEG) coated laccase.

8. The process as claimed in claim 1, wherein the aqueous solution further comprises at least one enzyme selected from catalase, glucose oxidase, or combinations thereof.

9. The process as claimed in claim 8, wherein the aqueous solution comprises laccase, catalase, and glucose oxidase; and activity ratio of laccase, catalase and glucose oxidase is in a range of 1.0:1.0:0.5 to 1.0:1.0:0.1.

10. The process as claimed in claim 1, wherein the mediator is selected from (2,2,6,6-tetra methyl piperidin-1-yl)oxyl (TEMPO), hydroxybenzotriazole (HOBT), dimethylglyoxime, N-hydroxy phthalimide (NHPI), 4-acetamido-TEMPO, TEMPO-4-amino-4-carboxylic acid (TOAC), 4-hydroxy-TEMPO, 4-hydroxy-TEMPO-benzoate, 4-oxo-TEMPO, 4-methoxy TEMPO, 4-amino-2,2,6,6,-tetramethylpiperidine, 4-amino-TEMPO or combinations thereof.

11. The process as claimed in claim 3, wherein the emulsifying agent is non-ionic surfactant selected from polyethylene glycol (PEG)-200, PEG-400, 2-[4-(2,4,4-trimethylpentan-2-yl) phenoxy]ethanol (triton-X 100), polyoxyethylene (80) sorbitan monooleate (tween 80), or combinations thereof; and the emulsifying agent is in a weight range of 0.1% to 5.0%.

12. The process as claimed in claim 1, wherein the oxygen source is selected from air, hydrogen peroxide, glucose, or combinations thereof.

13. The process as claimed in claim 6, wherein the extraction is carried out using a solvent selected from hexane, heptane, ethyl acetate, methyl tertiary butyl ether, methyl isobutyl ketone, or combinations thereof.

14. The process as claimed in claim 1, wherein the mole ratio of mediator and α-pinene is in a range of 1.0:5.0 to 1.0:15.0.

15. The process as claimed in claim 1, wherein the yield of the verbenone is in a range of 50% to 80%; and purity of verbenone is in a range of 95% to 99%.
